(19) United States
(12) Patent Application Publication
(10) Pub. No.: US 2023/0210639 A1
(43) Pub. Date: Jul. 6, 2023

(54) DENTAL SUPPORT SCREW

(71) Applicant: BIOMET 3i, LLC, Palm Beach Gardens, FL (US)

(72) Inventors: Kaushal Kumar, Palm Beach Gardens, FL (US); Jay Jayashankar, Warsaw, IN (US)

(21) Appl. No.: 18/091,124

(22) Filed: Dec. 29, 2022

Related U.S. Application Data
(60) Provisional application No. 63/295,765, filed on Dec. 31, 2021.

(51) Int. Cl. A61F 2/28; A61C 8/00

(52) U.S. Cl. CPC A61F 2/2846; A61C 8/0009

(57) ABSTRACT

An implantable screw for maintaining horizontal and/or vertical space during bone grafting procedures. The screw includes a head having a top surface configured to engage a dental membrane, a shaft, and a tip. The shaft is at least partially threaded to engage bone. The dental membrane and the screw provide space for bone growth material during bone grafting.

### CROSS-REFERENCE TO RELATED APPLICATIONS

This application claims the benefit of U.S. Provisional Application No. 63/295,765, titled “TENTING SCREW FOR DENTAL MEMBRANE” and filed on Dec. 31, 2021, which application is incorporated herein by reference in its entirety.

### TECHNICAL FIELD

The present disclosure relates to an implantable screw for maintaining horizontal and/or vertical space during a dental procedure such as, for example, ridge augmentation.

### BACKGROUND

Dental implants are commonly used as anchoring members in prosthodontics restorations to provide prosthetic teeth at one or more edentulous sites in a patient's dentition at which the patient's original natural teeth have been lost or damaged. Due to tooth decay, periodontal diseases, accidental injury, anatomical abnormalities, bone, gum and/or tooth loss, trauma, tumors, infections and other complications, a natural tooth of a patient may be removed or missing. As a result, dental implant devices may be implanted in the patient's bone structure to improve the patient's physical appearance and/or tooth function.

When an extracted or otherwise missing tooth is not immediately grafted or replaced with an implant, atrophy of the jawbone occurs over time and bone may begin to resorb. Consequently, individuals who have been partially edentulous for an extended period are left with an atrophic alveolar ridge that cannot securely support a dental implant/prosthesis.

In some cases, a ridge augmentation procedure is employed to add bone (e.g., bone height) to the jaw so that sufficient alveolar bone exists to place dental implants. With bone grafting-techniques for ridge augmentation, bone that has been lost may be built up again, therefore benefiting a patient's health and/or appearance by strengthening their jawbone and allowing more effective tooth replacement.

During a bone grafting procedure, a bone graft may be inserted into a socket in the patient's jaw and powder may be poured into any remaining void(s) between the bone graft and the socket. A membrane may then be sutured on top of the void. Conventional membranes may be formed from allograft or xenograft skin. However, a conventional membrane may not maintain its shape and therefore the membrane and the bone graft may deform when experiencing pressure. For example, a divot may form on the bone graft and the membrane when a patient chews food, thereby exerting pressure on the membrane and the bone graft.

Further, in some instances, depending on the bone void shape and location, an additional fixture may be used to contain the bone graft material within the bone void. For example, titanium mesh fixtures have been used to contain bone graft material within a bone void during a bone augmentation. However, the fixture, e.g., a titanium mesh, is a foreign body and can cause various issues and is required to be removed after the bone augmentation. Thus, the fixture requires a patient to undergo a further procedure. The removal of these fixtures can cause tissue disruption that undesirably exposes new bone and disrupts vascularity leading to associated complications or can be difficult to remove due to bone overgrowth. The removal of these fixtures can cause tissue disruption that undesirably exposes new bone and disrupts vascularity leading to associated complications or can be difficult to remove due to bone overgrowth.

### SUMMARY

The present disclosure provides a dental support screw for creating and/or maintaining a space or void during a dental procedure and/or during bone growth and recovery. Often, the head of the support screw is placed away from the bone, providing enough space for placement of bone graft material and subsequent bone growth. Conventional implantable screws such as a tenting-type screw in this case have commonly been made of surgical quality metals, such as stainless steel, and surgical grade titanium and titanium alloys. Other commonly used materials include ceramics and various plastics. However, the use of these materials have many drawbacks. For example, metal screws typically remain in the body unless they are later removed by a separate operation. Further, metal screws can potentially irritate tissue in close proximity to the screw, shed harmful ions into the body, or loosen to the point of injury among other things. Other commonly used materials offer a comparable list of drawbacks.

In some embodiments, new implantable devices comprising screws formed from bone material and/or artificial bone material are provided for maintaining proper spacing during bone grafting in certain dental regenerative procedures. In some embodiments, the screw comprises cortical bone material and can become incorporated into, for example, a newly generated alveolar ridge, thereby reducing or eliminating the need to have the screw removed prior to the placement of dental implant.

In some embodiments, the contoured-shaped head of the screw ensures an interface between the gingiva and the screw, minimizing the likelihood of dehiscence and creating greater space for the bone growth.

In various embodiments, an implantable device for maintaining space during bone grafting procedures is provided comprising at least one screw, the screw comprising a contoured head, a threaded shaft and a tip adapted to penetrate bone tissue, wherein the screw comprises allograft bone material.

In various embodiments, an implantable device is provided for maintaining space during bone grafting procedures comprising a screw wherein the screw comprises a contoured head, a threaded shaft and a tip adapted to penetrate bone tissue.

In various embodiments, an implantable device is provided that can be used in conjunction with a method of grafting bone in oral procedures. The method comprising, implanting into the jawbone an implantable device wherein an implantable device comprises a first screw comprising a contoured head, a threaded shaft and a tip adapted to penetrate bone tissue. The method includes attaching a dental membrane over the implantable device.

These and other examples, advantages, and features of the present dental membranes will be set forth in part in the following Detailed Description and the accompanying drawings. This Overview is intended to provide non-limiting examples of the present subject matter—it is not intended to provide an exclusive or exhaustive explanation. The Detailed Description and drawings are included to provide further information about the present porous metal dental implants.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

These and other examples, advantages, and features of the present dental membranes will be set forth in part in the following Detailed Description and the accompanying drawings. This Overview is intended to provide non-limiting examples of the present subject matter—it is not intended to provide an exclusive or exhaustive explanation. The Detailed Description and drawings are included to provide further information about the present porous metal dental implants.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 illustrates a perspective view of a jaw with insufficient bone for implant placement;

FIG. 2 illustrates a cross-sectional view of a dental support screw according to at least one embodiment of the present disclosure;

FIG. 3 illustrates a cross-sectional view of the dental support screw being used to maintain space between the bone of the patient and a dental membrane according to at least one embodiment of the present disclosure;

FIG. 4 illustrates a cross-sectional view of the tenting screw being used to maintain space between the bone of the patient and a dental membrane according to at least one embodiment of the present disclosure; and

FIG. 5 illustrates a flowchart according to at least one embodiment of the present disclosure.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The present disclosure provides an implantable device, e.g., a support screw, that can be used during a dental procedure such as, for example, a ridge augmentation procedure. In some embodiments, the implantable device or support screw can be used with a dental membrane, bone graft materials, or the like. The support screw is able to maintain space (vertically and/or horizontally) during bone grafting procedures. The support screw (also referred to herein as “screw”) includes a contoured head, a shaft, and a tip adapted to penetrate bone tissue. A portion of the shaft can be threaded. The contoured head may have a region adapted to support, for example, the dental membrane. In some embodiments the support screw is formed from bone material. In other embodiments, the support screw is formed from artificial bone material with characteristics of bone material.

The support screw of the present disclosure beneficially does not need to be removed surgically after healing and, e.g., before a dental implant is installed within the patient's mouth. Since the support screw is formed form bone material or artificial bone material, the support screw will incorporate into the regenerated bone. As the support screw is formed from bone or artificial bone material, the tenting screw will become integral with the surrounding bone.

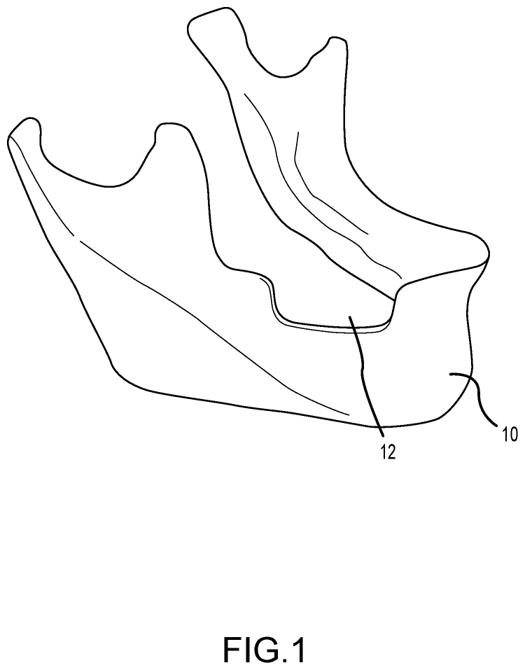

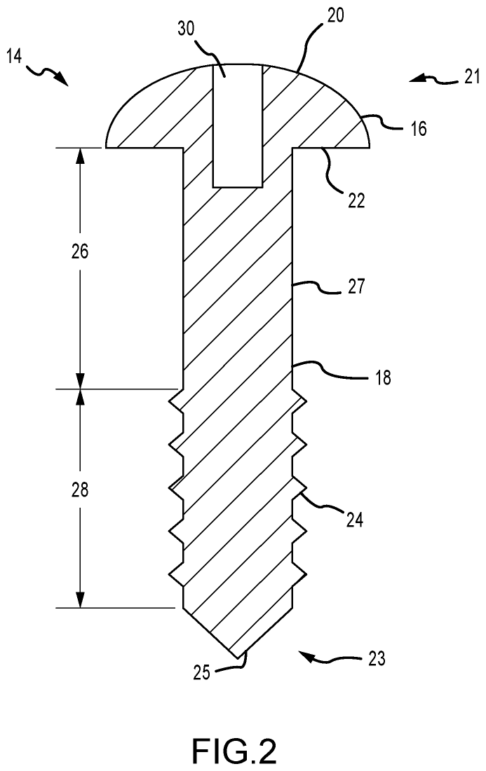

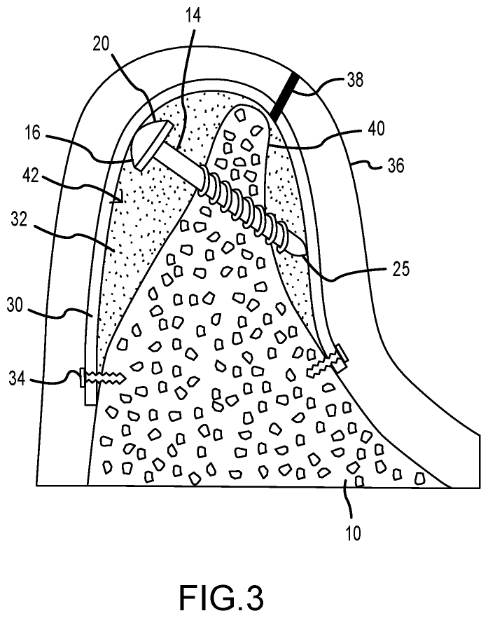

FIG. 1 shows a patient jaw 10 having insufficient bone at a surgical site 12. The insufficient bone in the patient jaw 10 may be due to factors such as periodontal disease, lost teeth, bone resorption, a thin maxillary and mandibular arch due to genetics, etc. The surgical site 12 can vary between patient's and can have a variety of shapes and sizes. During bone regeneration to form a dental ridge sufficient to receive a dental implant, the shape and size of the regenerated bone is important. A bone void surrounded by four walls may be easier to contain bone graft material 32 (shown in FIG. 3) compared to a bone void only having two or the four walls. As described herein, a support screw 14 (shown in FIGS. 2-4) can maintain the space needed to fill the bone void with the bone graft material 32 and a dental membrane 30 (shown in FIG. 3) can be used to contain the bone graft material 32. In embodiments where the dental membrane 30 is used to contain the bone graft material 32 and/or cover the bone void, the support screw 14 may also provide support to the dental membrane 30.

FIG. 2 illustrates a cross-sectional view of the support screw 14. As discussed herein, the support screw 14 is formed of bone material, e.g., cortical bone material. Cortical bone is dense and forms the surface of bones. The cortical bone can be obtained from human autologous bone and/or allograft tissue. Autologous and/or allograft tissues are biocompatible, are strong, and biointegrate in time with the recipient patient's tissue and can be shaped into a screw commercially in a manufacturing environment. The cortical bone can be provided in a sterile form. In some embodiments, the cortical bone can be mineralized. In other embodiments, the bone can be reconstituted, milled, and/or demineralized.

It will be understood by those of ordinary skill in the art that the cortical bone material may be obtained from non-human sources (e.g., bovine, ovine, porcine, or the like), such as for example, xenograft material from animal-derived bone tissue. In additional to bone material, the screw 14 can be formed from artificial bone materials that have similar characteristics of cortical bone.

As seen in FIG. 2, the support screw 14 extends from a coronal end 21 to an apical end 23. The support screw 14 includes a head 16, a shaft 18, and a tip 25. At least a portion of the shaft 18 includes threads 24, which enable the support screw 14 to be anchored into the jaw. More specifically, the shaft 18 includes a threaded portion 28 and a non-threaded portion 26. However, in some examples, the entire shaft 18 can be threaded. In some embodiments the tip 25 is pointed and configured to penetrate bone. As discussed herein, the support screw 14 is formed of bone material (e.g., cortical bone) or alternative bone material that imitates cortical bone such that the support screw 14 will become integrated into the bone as it grows. The integration of the support screw 14 into the newly forming bone tissue reduces the need to remove the support screw 14 as is normally the case with conventional screws of other compositions.

In various examples, the head 16 is contoured. For example, the top surface 20 of the head 16 can be concave as illustrated, which can support the dental membrane 30 and reduce the risk of damaging the dental membrane 30 and/or the soft tissue of the patient. As also shown in the illustrated embodiment, a bottom surface 22 of the head 16 of the support screw 14 can be flat. In other embodiments, the bottom surface 22 can be curved or any other shape. The screw 14 can also include an engagement bore 30 that is configured to non-rotationally or rotationally couple with a tool that is used to insert the screw 14 into the bone of the patient. In other embodiments, the support screw 14 may include engagement surfaces on the head 16 of the screw such that, for example, a wrench can engage the engagement surfaces to screw the support screw 14 into the bone of the patient.

The support screw 14 can have a porosity of about 5% to about 10%. In other embodiments the support screw 14 can have porosity less than 5% or greater than 10%. It will also be appreciated that the porosity of the support screw 14 can change along a longitudinal axis extending from the coronal end 21 to the apical end 23. In still other embodiments the shaft 18 can have a porosity different from the head 16. The support screw 14 can also have a pore diameter of about 10 microns to about 50 microns. In other embodiments the support screw 14 can have a pore diameter less than 10 microns or greater than 50 microns.

After installation, the screw head 16 provides sufficient space that is maintained or created for grafting areas of the, for example, alveolar ridge, allowing the bone graft material 32 to either induce new bone formation or serve as an osteoconductive scaffold as bone forms in the space provided. As discussed herein, the position of the support screw 14 can vary and depend on whether only vertical, only horizontal, or both spacing is needed during the bone augmentation. Further, it will be appreciated that more than one support screw 14 can be used at the surgical site 12. For example, a first support screw can be positioned adjacent to a second support screw. In another example, a first support screw can be used to maintain a vertical space in the bone void and a second support screw can be used to maintain a horizontal space in the bone void. Thus, any number of support screws 14 can be positioned anywhere in the surgical site 12.

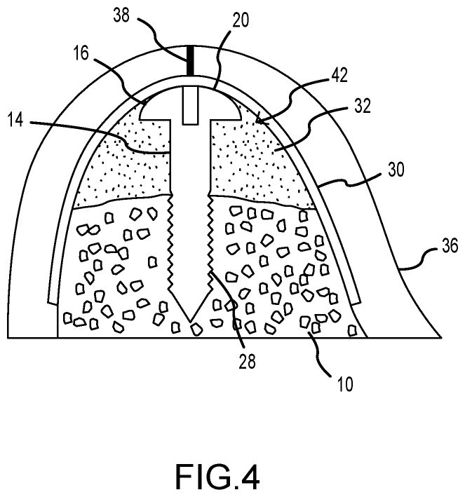

FIG. 3 illustrates the support screw 14 being used to provide both vertical and horizontal space for the bone augmentation. FIG. 4 illustrates the support screw 14 being used for vertical space for the bone augmentation.

As seen in FIG. 3, the support screw 14 is inserted through a portion of a bone ridge 40. The top surface 20 of the head 16 of the support screw 14 contacts the dental membrane 30. The tip 25 of the support screw 14 extends through the portion of the bone ridge 40 and is in contact with the dental membrane 30. The space or void 42 created by the support screw 14 and the dental membrane 30 is filled with bone graft material 32. In other words, the support screw 14 can support the space or void 42 to enable the bone graft material 32 to fill the space or void 42. The dental membrane 30 can be secured to the patient via, for example, membrane tacking screws 34 or sutures. Once the bone graft material 32 is secured within the dental membrane 30, the soft tissue 36 of the patient can be closed via a suture 38.

As seen in FIG. 4, the threaded portion 28 of the support screw 14 is positioned within the bone 10. The top surface 20 of the head 16 of the support screw 14 contacts the dental membrane 30 and forms or defines the space or void 42. The bone graft material 32 can be added to fill the space or void 42. The dental membrane 30 can be secured to the patient and the soft tissue 36 of the patient can be close via a suture 38. The support screw 14 helps support the dental membrane 30 and maintain the space 42 and also helps prevent damage or deformations to the dental membrane 30 and/or the space 42.

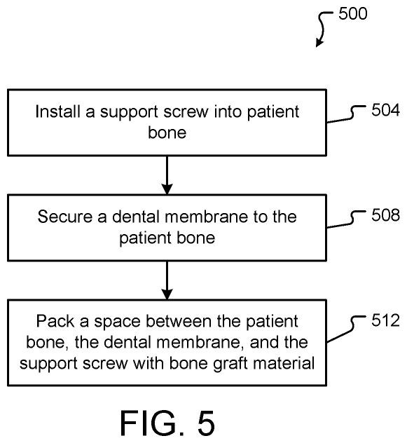

Turning to FIG. 5, a flowchart for a method 500 of performing a bone augmentation procedure using a support screw such as the support screw 14 is provided.

The method includes installing a support screw such as the support screw 14 into patient bone in a bone void along a surgical site such as the surgical site 12 at step 504. The support screw may be formed from bone material. As previously described, the support screw may comprise a head such as the head 16 have a top surface such as the top surface 20 configured to support a dental membrane such as the dental membrane 30. The support screw may also comprise a shaft such as the shaft 18 extending from a bottom surface such as the bottom surface 22 of the head. The shaft may comprise a threaded portion such as the threaded portion 28 and an unthreaded portion such as the unthreaded portion 26. It will be appreciated that in some embodiments the shaft may not include the unthreaded portion and the entire shaft may be threaded. The support screw also comprises a tip configured to engage the patient bone.

The method 500 also comprises securing the dental membrane to the patient bone at step 508. As previously described, the support screw may support the dental membrane and prevent deformation or damage to the dental membrane while maintaining a space such as the space 42 in the bone void. The dental membrane may be configured to protect the bone void.

The method also comprises packing the space between the bone, the dental membrane, and the support screw with bone grafting material such as the bone grafting material 42 at step 512. The bone grafting material may be configured to promote bone growth in the bone void.

As previously described, because the support screw is formed from bone material or artificial material with characteristics of bone, after a healing period the support screw does not need to be removed from the patient. In such instances, the support screw has become integral with regenerated bone at the surgical site.

It will be appreciated that in some embodiments the step 512 may be performed prior to the step 508.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

### EXAMPLES

To illustrate the dental membrane, a non-limiting list of examples is provided here:

In Example 1, a screw for maintaining space during a bone augmentation procedure, the screw comprising: a head having a top surface configured to support a dental membrane; a shaft extending from a bottom surface of the head, as least a portion of the shaft having threads; and a tip, wherein the screw is formed from bone material.

Example 2 can include, or can optionally be combined with the subject matter of Example 1, to optionally include wherein the bone material is selected from one of allograft and xenograft.

Example 3 can include, or can optionally be combined with the subject matter of one or any combination of Examples 1 or 2 to optionally include wherein the bone material is an artificial bone material that has characteristics of cortical bone.

Example 4 can include, or can optionally be combined with the subject matter of one or any combination of Examples 1 through 3 to optionally include wherein screw has a porosity of about 5 percent (%) to about 10%.

Example 5 can include, or can optionally be combined with the subject matter of one or any combination of Examples 1 through 4 to optionally include wherein the screw has a pore diameter of about 10 microns (μm) to about 50 μm.

Example 6 can include, or can optionally be combined with the subject matter of one or any combination of Examples 1 through 5 to optionally include wherein the porosity of the screw changes along a longitudinal axis of the screw.

Example 7 can include, or can optionally be combined with the subject matter of one or any combination of Examples 1 through 6 to optionally include wherein the shaft has a porosity that is different from the porosity of the head of the screw.

Example 8 can include, or can optionally be combined with the subject matter of one or any combination of Examples 1 through 7 to optionally include wherein the portion of the shaft that includes the threads is the entire shaft.

Example 9 can include, or can optionally be combined with the subject matter of one or any combination of Examples 1 through 8 to optionally include wherein the portion of the shaft that includes the threads is about 75 percent (%) of a total length of the shaft.

In Example 10, a method of performing a bone augmentation procedure, the method including: installing a screw into a bone of the patient along a surgical site, the screw including: a head having a top surface configured to support a dental membrane; a shaft extending from a bottom surface of the head, as least a portion of the shaft having threads; and a tip, wherein the screw is formed from bone material; securing a dental membrane to the bone of the patient such that a head of the screw supports the dental membrane; and packing the space between the bone, the dental membrane, and the screw with bone grafting material.

Example 11 can include, or can optionally be combined with the subject matter of Example 10, wherein the first layer and the second layer are formed integrally with each other.

Example 12 can include, or can optionally be combined with the subject matter of one or any combination of Examples 10 through 11 to optionally include wherein, after the healing period, the screw has become integral with regenerated bone at the surgical site.

Example 13 can include, or can optionally be combined with the subject matter of one or any combination of Examples 10 through 12 to optionally include wherein the bone material is selected from one of allograft and xenograft.

Example 14 can include, or can optionally be combined with the subject matter of one or any combination of Examples 10 through 13 to optionally include wherein the bone material is an artificial bone material that has characteristics of cortical bone.

Example 15 can include, or can optionally be combined with the subject matter of one or any combination of Examples 10 through 14 to optionally include wherein screw has a porosity of about 5% to about 10%.

Example 16 can include, or can optionally be combined with the subject matter of one or any combination of Examples 10 through 15 to optionally include wherein the screw has a pore diameter of about 10 microns (μm) to about 50 μm.

Example 17 can include, or can optionally be combined with the subject matter of one or any combination of Examples 10 through 16 to optionally include wherein the porosity of the screw changes along a longitudinal axis of the screw.

Example 18 can include, or can optionally be combined with the subject matter of one or any combination of Examples 10 through 17, to optionally include wherein the shaft has a porosity that is different from the porosity of the head of the screw.

Example 19 can include, or can optionally be combined with the subject matter of one or any combination of Examples 10 through 18, to optionally include wherein the portion of the shaft that includes the threads is about 50% to about 75% of a total length of the shaft.

Example 20 can include, or can optionally be combined with the subject matter of one or any combination of Examples 10 through 18, to optionally include wherein the portion of the shaft that includes a total length of the shaft.

In Example 21, a support screw for maintaining space during a bone augmentation procedure, the screw comprising: a head having a concave top surface configured to support a dental membrane, the head comprising an engagement bore configured to engage with a tool to insert the support screw into patient bone; a shaft extending from a bottom surface of the head, as least 50 percent (%) of the shaft having threads; and a tip configured to engage the patient bone, wherein the support screw is formed from bone material.

Example 22 can include, or can optionally be combined with the subject matter of Example 21, to optionally include wherein the bone material is selected from one of allograft and xenograft.

Example 23 can include, or can optionally be combined with the subject matter of one or any combination of Examples 21 or 22 to optionally include wherein the bone material is mineralized.

Example 24 can include, or can optionally be combined with the subject matter of one or any combination of Examples 21 through 23 to optionally include wherein the shaft has a porosity that is different from the porosity of the head of the screw.

Each of these non-limiting examples can stand on its own, or can be combined in various permutations or combinations with one or more of the other examples

What is claimed is:

1. A screw for maintaining space during a bone augmentation procedure, the screw comprising:
a head having a top surface configured to support a dental membrane;
a shaft extending from a bottom surface of the head, as least a portion of the shaft having threads; and
a tip,
wherein the screw is formed from bone material.

2. The screw of claim 1, wherein the bone material is selected from one of allograft and xenograft.

3. The screw of claim 1, wherein the bone material is an artificial bone material that has characteristics of cortical bone.

4. The screw of claim 3, wherein the screw has a porosity of about 5 percent (%) to about 10%.

5. The screw of claim 3, wherein the screw has a pore diameter of about 10 microns (μm) to about 50 μm.

6. The screw of claim 1, wherein the porosity of the screw changes along a longitudinal axis of the screw.

7. The screw of claim 1, wherein the shaft has a porosity that is different from the porosity of the head of the screw.

8. The screw of claim 1, wherein the portion of the shaft that includes the threads is the entire shaft.

9. The screw of claim 1, wherein the portion of the shaft that includes the threads is about 75 percent (%) of a total length of the shaft.

10. A support screw for maintaining space during a bone augmentation procedure, the screw comprising:
a head having a concave top surface configured to support a dental membrane, the head comprising an engagement bore configured to engage with a tool to insert the support screw into patient bone;
a shaft extending from a bottom surface of the head, as least 50 percent (%) of the shaft having threads; and
a tip configured to engage the patient bone,
wherein the support screw is formed from bone material.

11. The support screw of claim 10, wherein the bone material is selected from one of allograft and xenograft.

12. The support screw of claim 10, wherein the bone material is mineralized.

13. The support screw of claim 10, wherein the shaft has a porosity that is different from the porosity of the head of the screw.

14. A method of performing a bone augmentation procedure, the method including:
installing a screw into a bone of the patient along a surgical site, the screw including:
a head having a top surface configured to support a dental membrane,
a shaft extending from a bottom surface of the head, as least a portion of the shaft having threads; and
a tip,
wherein the screw is formed from bone material;
securing a dental membrane to the bone of the patient such that a head of the screw supports the dental membrane; and
packing the space between the bone, the dental membrane, and the screw with bone grafting material.

15. The method of claim 14, wherein the bone material is selected from one of allograft and xenograft.

16. The method of claim 14, wherein the bone material is an artificial bone material that has characteristics of cortical bone.

17. The method of claim 14, wherein the screw has a porosity of about 5% to about 10%.

18. The method of claim 14, wherein the screw has a pore diameter of about 10 microns (μm) to about 50 μm.

19. The method of claim 17, wherein the porosity of the screw changes along a longitudinal axis of the screw.

20. The method of claim 14, wherein the shaft has a porosity that is different from the porosity of the head of the screw.
